(19) United States
(12) Patent Application Publication
(10) Pub. No.: US 2026/0151252 A1
(43) Pub. Date: Jun. 4, 2026

(54) NECK COLLAR AND METHOD OF MANUFACTURE

(71) Applicant: UCL BUSINESS LTD, London (GB)

(72) Inventors: Deepak KALASKAR, London (GB); Abir DUTTA, London (GB)

(21) Appl. No.: 19/406,140

(22) Filed: Dec. 2, 2025

Related U.S. Application Data
(63) Continuation of application No. PCT/GB2024/051405, filed on May 30, 2024.

(30) Foreign Application Priority Data
Jun. 2, 2023 (GB) 2308318.1

(51) Int. Cl. A61F 5/055; A41D 13/05; B33Y 50/00; B33Y 80/00

(52) U.S. Cl. CPC A61F 5/055; A41D 13/0512; B33Y 50/00; B33Y 80/00

(57) ABSTRACT

A custom neck collar is provided, for supporting the neck and head of a user in a pre-defined position. The custom neck collar comprises: a three-dimensional (3D) printed collar body having an inner surface that conforms to the contours of a user's neck area, and wherein at least a portion of the collar body is formed of or comprises a 3D mesh structure.

### TECHNICAL FIELD

This invention relates generally to wearable orthotic devices, particularly to bespoke custom fit neck collars and their method of manufacture.

### BACKGROUND TO THE INVENTION

Orthoses are externally applied devices that provide structural support or functional adjustment to the body. They are intended to improve the user's level of function and quality of life by guiding, restricting or supporting movement. Traditionally, custom orthoses are made via a laborious, manual process by skilled clinicians, involving casting, sculpting, and moulding of a thermoplastic and fitting to the body. This results in waiting times of up to 6 weeks, with multiple patient visits, at a social and economic cost, and no guarantee of patient concordance. Orthotics may have to be revised regularly, requiring the orthosis to be re-made and repetition of this time-consuming and skilled process. The application and combination of new technologies may help to overcome the limitations of these traditional approaches while replicating or improving the mechanical, functional and aesthetic properties.

Additive manufacturing, or three-dimensional (3D) printing, is a process whereby material is sequentially added to print a 3D object from a computer model. It is becoming an accessible and widespread technology, which has demonstrated numerous applications within medicine, from surgical planning, education, prostheses and drug delivery. 3D printed orthotic devices can be made to have comparable biomechanical properties to traditionally manufactured devices, with potential for fine control over these properties. Orthotic designs made via computer-aided design (CAD), can be widely distributed through large open-source communities and then customised by the end-user, allowing adjustment of the resulting model to meet the user needs. This contrasts with manufactured, commercial devices—a ‘one-size-fts-all’ approach—and greatly reduces the cost associated with creating customised, bespoke devices.

Cervical or neck collars are used for three functions: to stabilise or immobilise the neck following trauma or surgery; to support the neck in cases of chronic neck pain; or to hold the head up in neuromuscular weakness (known as neck-drop syndrome). Neck collars are intentionally padded for comfort, and generally comprise a hard outer shell with a padded (typically foam) liner attached to the inner surface of the outer shell.

Current state-of-the-art processes for manufacturing bespoke neck collar involve a combination of 3D scanning and 3D printing. In general, 3D scans of the patient's neck area (e.g. using a 3D laser scanner) are used to create an accurate digital 3D model of patient's the neck area, from which a 3D printable geometry of the outer shell of the bespoke neck collar is produced. A separate padded liner is then fitted to the inner surfaces of the 3D printed shell to complete the final neck orthoses. For example, this approach is described in Hale et al. “A digital workflow for design and fabrication of bespoke orthoses using 3D scanning and 3D printing, a patient-based case study” published in Nature Scientific Reports, 10, 7028 (2020). In this work, the 3D collar geometry is used to produce a 2D template for cutting a customised foam liner that, when fitted, conforms without distortion to the 3D inner surface of the shell.

Current solutions for 3D printed neck collars focus on providing a bespoke anatomically shaped collar for the patient. However, the usage of cushioned linings beneath the hard collar shell obstructs the breathability, promotes sweating and are unhygienic for prolonged use.

The present invention seeks to solve this problem by providing a breathable, flexible, hygienic for prolonged usage lining-free bespoke cervical orthoses device for patients with severe to moderate head and neck disorders.

### SUMMARY OF THE INVENTION

According to a first aspect, there is provided a custom neck collar for supporting the neck and head of a user in a pre-defined position, comprising:
a three-dimensional (3D) printed collar body having an inner surface that conforms to the contours of a user's neck area, and wherein at least a portion of the collar body is formed of or comprises a 3D mesh structure.

The at least a portion comprising the 3D mesh structure may form at least part of the inner surface of the collar body for cushioning. The 3D mesh structure may be provided at one or more pressure points of the neck area.

At least a portion comprising the 3D mesh structure may form the entire inner surface of the collar body for cushioning.

The collar body may comprise a outer shell portion and at least one inner cushioning portion comprised of the 3D mesh structure. The at least one inner cushioning portion may be unitary with the outer shell portion. The outer shell portion and the at least one inner cushioning portion may be formed of or comprise different polymer materials.

The 3D mesh structure of the at least one inner cushioning portion may be formed of or comprise a polymer material and may be configured to be relatively soft, compressible and/or pliable compared to the outer shell portion. The 3D mesh structure may be formed of or comprise an elastomeric polymer material, or a non-elastomeric polymer material.

The outer shell portion may be formed of or comprise a polymer material and may be configured to be:
substantially rigid to restrict movement of the neck and head from the predefined position, or
substantially flexible to permit limited movement of the neck and head from the predefined position.

The outer shell portion may comprise one or more openings for ventilation. The 3D mesh structure of the at least one inner cushioning portion may extend across at least some of the one or more openings.

The entire collar body may be is formed of or comprise the 3D mesh structure.

The outer shell portion may also be comprised of the 3D mesh structure. The 3D mesh structure of the outer shell portion and the 3D mesh structure of the at least one inner cushioning portion may be defined by a plurality of mesh parameters. At least one mesh parameter of the outer shell portion may be different to the corresponding at least one mesh parameter of the at least one inner cushioning portion.

The outer shell portion may be a solid outer shell portion without a 3D mesh structure.

The 3D mesh structure may be formed of or comprise an antibacterial material.

The collar body may be configured to be:
substantially flexible to permit limited movement of the neck and head from the predefined position; or
substantially rigid to restrict movement of the neck and head from the predefined position.

The 3D mesh structure may be an open cell structure.

The 3D mesh structure may comprise one of the following geometries: a gyroid lattice structure, a hexagonal lattice structure, a Voronoi lattice structure, a rhombohedral lattice structure, a dodecahedron lattice structure, and a Schwartz lattice structure.

The collar may further comprise one or more sensors integrated on or in the collar body. The one or more sensors may be configured to monitor one or more of: a force or pressure exerted on the collar by the user's neck, a temperature and/or a humidity in the collar.

The collar body may comprise an anterior first portion and a second portion that are connectable to permit transition of the collar between an open and closed position for fitting to a user. The first and second parts may be:
(i) connected at a first side of the collar body by a hinge or hinge portion and connectable at a second side of the collar body by one or more releasable fastening devices; or
(ii) connectable at first and second sides of the collar body by one or more releasable fastening devices.

The one or more releasable fastening devices may be or comprise one or more of the following: a hook and loop fastener, a magnetic fastener, and a mechanical fastener.

The collar may not comprise additional cushioning material or a separate padded liner.

The collar body may be formed of or comprise one or more polymer materials.

According to a second aspect, there is provided a method of manufacturing a custom neck collar for supporting the neck and head of a user in a pre-defined position, comprising:
generating a solid model of a custom neck collar body with an inner surface that conforms to the contours of a user's neck area and with at least a portion of the neck collar body formed of a three-dimensional (3D) mesh structure, based on 3D surface scan data of the user's neck area obtained whilst their neck and head is in the predefined position and one or more input mesh parameters; and
fabricating the custom neck collar body based on the solid model, at least in part, by using a 3D printer.

The one or more input mesh parameters may include one or more of the following: a type of unit cell, an infill density, a coverage and/or perimeter of the mesh structure, and a width of beams of the mesh structure.

The step of generating the solid model may comprise:
generating an input solid model of a solid neck collar body having an inner surface that conforms to the contours of a user's neck area based on 3D surface scan data of the user's neck area obtained whilst their neck and head is in the predefined position; and
converting at least one region of the input solid model adjacent the inner surface into a 3D mesh structure based on the one or more input mesh parameters to provide an output solid model of a collar body with an outer shell portion and at least one inner cushioning portion comprised of the 3D mesh structure, wherein the 3D mesh structure of the inner cushioning portion is configured, via the one or more mesh parameters, to be relatively soft, compressible and/or pliable compared to the outer shell portion.

The solid model of the neck collar body may be formed completely of the 3D mesh structure.

The step of generating the solid model may comprise:
converting the one or more regions of the input solid model adjacent the inner surface into a 3D mesh structure based on the one or more first input mesh parameters to provide at least one inner cushioning portion comprised of a first 3D mesh structure; and
converting the rest of the input solid model into a 3D mesh structure based on one or more second input mesh parameters to provide an output solid model of a collar body with an outer shell portion and at least one inner cushioning portion comprised of a 3D mesh structure.

Generating the input solid model of the neck collar body may comprise:
creating a scan mesh defining a 3D surface that conforms to the 3D surface scan data;
relaxing the scan mesh away from the 3D surface scan data, the relaxed scan mesh defining a relaxed 3D surface; and
extruding the relaxed 3D surface to a desired thickness of the neck collar body to produce the input solid model.

The custom neck collar body may be fabricated entirely using a 3D printer. Fabricating the custom neck collar body may comprise: 3D printing a solid neck collar body; and forming the 3D mesh structure in the at least a portion of the 3D printed solid neck collar body using subtractive manufacturing.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the device may have corresponding features definable with respect to the method(s), and vice versa, and these embodiments are specifically envisaged.

### BRIEF DESCRIPTION OF DRAWINGS

In order that the invention can be well understood, embodiments will now be discussed by way of example only with reference to the accompanying drawings, in which:

FIG. 1A shows representative diagram of a custom neck collar comprising an integrated mesh cushioning layer according to an embodiment of the invention;

FIG. 1B shows the integrated mesh cushioning layer of FIG. 1A, the inset shows an enlarged view of the mesh structure;

FIGS. 1C and 1D show example side and rear views of the collar of FIG. 1 fitted to a patient;

FIG. 2A shows representative diagram of a custom neck collar comprising an integrated mesh cushioning layer according to another embodiment of the invention;

FIG. 2B shows the integrated mesh cushioning layer of FIG. 2A, the inset shows an enlarged view of the mesh structure;

FIG. 3A shows representative diagram of a custom neck collar formed entirely of a mesh structure according to another embodiment of the invention;

FIG. 3B shows an enlarged view of the mesh structure of FIG. 3A;

FIG. 4 shows representative diagram of a custom neck collar formed entirely of a mesh structure according to another embodiment of the invention;

FIG. 5 shows a schematic block diagram of a neck collar according to an embodiment of the invention including one or more sensors;

FIG. 6A shows a representative diagram of a custom neck collar comprising plurality of integrated sensors located as various anatomical landmarks;

FIG. 6B shows a schematic cross-section the neck collar of FIG. 6A at a location of a sensor;

FIG. 7 shows a representative diagram of a custom neck collar according to another embodiment of the invention comprising a hinge portion and a fastening for fitting the collar to a patient;

FIG. 8 shows a schematic diagram of a method of manufacturing the custom neck collar comprising mesh structures; and

FIGS. 9A-9D show representative diagrams of various steps of the method of FIG. 5.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

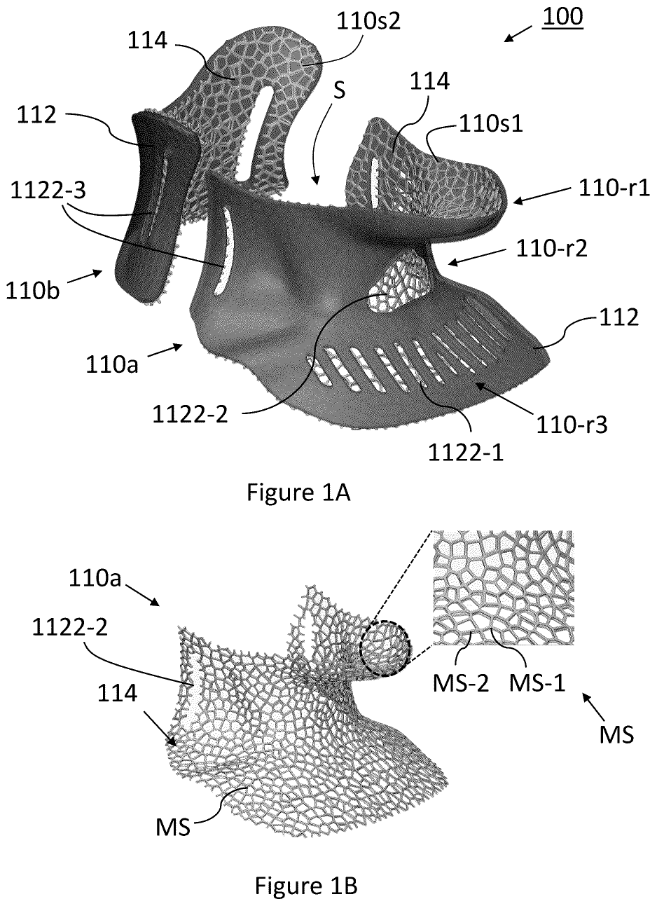

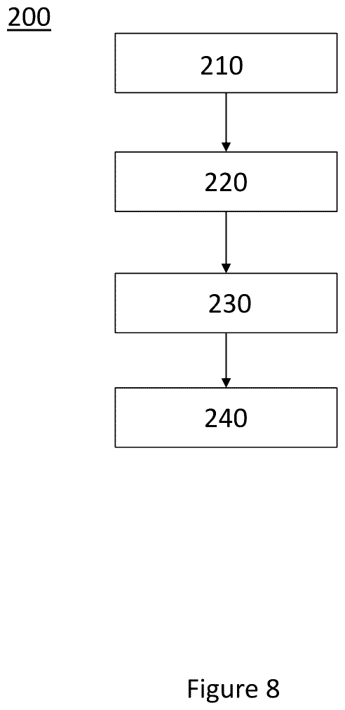

FIG. 1A shows a representative diagram of a custom neck collar 100 according to an embodiment of the invention. The collar 100 comprises a customised collar body 110 having an inner surface 110s that conforms to the contours of a user's neck area. The collar body 110 comprises a solid outer shell portion 112 and an inner cushioning portion 114 that forms or extends over the inner surface 110s. Unlike conventional approaches, the inner cushioning portion 114 is integrated with the outer shell portion 112 and is comprised of a three-dimensional (3D) mesh structure MS. Specifically, the inner cushioning portion 114 and the outer shell portion 112 are unitary and formed as a single object from one or more materials using 3D printing (additive manufacturing) based on 3D scans of the user's neck area, as will be described in more detail below with reference to the method 200 of FIG. 8.

The 3D mesh structure MS of the cushioning portion 114 is shown in more detail in FIG. 1B, in which the outer shell portion 112 is omitted for clarity. The 3D mesh structure MS is an open cell structure configured to be substantially soft, compressible and/or pliable to provide an integrated breathable cushioning layer that removes the requirement of a separate foam or fabric cushioned/padded lining and the associated hygiene issues that is commonplace in conventional neck collars. Meanwhile, the outer shell portion 112 is configured to be relatively hard and/or rigid compared to the inner cushioning portion 114 to provide the required support and stability to the user's neck and head (although a degree of flexibility is permitted for fitting and certain applications, as described below).

The collar body 110 is formed of or comprises one or more 3D printable polymer materials. The 3D mesh structure MS of the cushioning portion 114 is preferably formed of or comprises a soft/elastomeric polymer material for added comfort, such as a silicone rubber. However, the 3D mesh structure MS of the cushioning portion 114 can instead be formed of or comprise a more rigid or semi-rigid polymer material, such polypropylene or polyurethane-based materials, whereby the desired substantially soft, compressible and/or pliable properties of the cushioning portion 114 can be produced by the geometric properties of the 3D mesh structure MS itself (discussed in more detail below). Preferably, the polymer material of the 3D mesh structure MS also has antibacterial properties. In this case the polymer material can be an antibacterial polymer with natural antibacterial properties or comprise antibacterial agents/additives, as is known in the art. 3D printable polymer materials with ISO standard (ISO 22196:2011) antibacterial properties are commercially available. Commonly used antibacterial additives used in these polymers include silver, silver nitrates, silver oxides, and copper. In this way, the neck collar 110 is configured to be easily washable/cleanable and is suitable for hygienic prolonged use. The outer shell portion 112 is is formed of or comprises a relatively hard polymer material compared to the cushioning portion 114, such as nylon, acrylic styrene acrylonitrile, polylactic acid or polycarbonate.

The outer shell portion 112 can be configured, depending on the application, to be substantially rigid to restrict movement of the neck and head from the predefined position, or to be substantially flexible or semi-rigid to permit limited movement of the neck and head from the predefined position. A more rigid neck collar 100 is suitable for use as a custom fit cervical orthosis for trauma patients that require neck and head support with limited mobility. A more flexible neck collar 100 is suitable for use in injury recovery as a custom fit cervical orthosis for non-trauma patients that require support with a degree of mobility, and in injury prevention as a custom fit protective collar, e.g. for aircraft pilots, racing car drivers or the like that may suffer neck injuries as a result of impacts, jolts or high g-force. It will be appreciated that the rigidity or flexibility of the outer shell portion 110 is influenced by a number of properties, including: the mechanical properties of the polymer material used, the thickness of the outer shell portion 112, and the presence, location and geometry of any openings 1122 (see below). For example, based on the design consideration and patient's condition (e.g. the degree of support required), the typical thickness of the outer shell portion 112 may vary within the range of approximately 3 to 5 mm. The extrinsic properties of the outer shell portion 112 can therefore be controlled through at least these parameters to suit the specific application and user requirements.

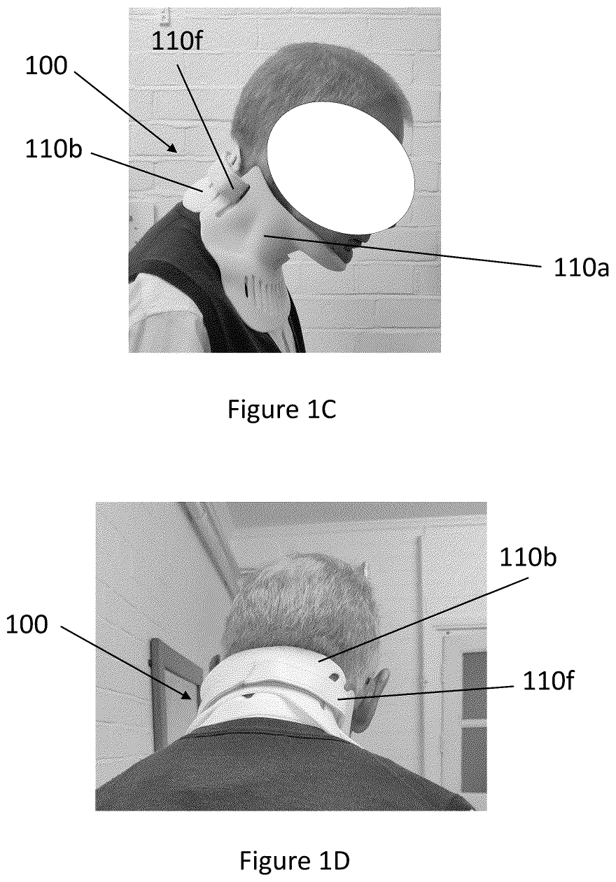

The 3D mesh structure MS is characterised by a 3D distribution of points MS-1 connected by struts or beams MS-2, as illustrated in the inset to FIG. 1B. The 3D mesh structure MS comprises a unit cell which is repeated in three dimensions. In embodiment of FIGS. 1A and 1B, the 3D mesh structure is a Voronoi lattice structure, but other open cell structures can be used including, but not limited, to a gyroid lattice structure, a hexagonal lattice structure, a rhombohedral lattice structure, a dodecahedron lattice structure, and a Schwartz lattice structure (e.g. see FIG. 2A). It will be appreciated that the softness and/or mechanical properties of the 3D mesh structure MS are determined by and can be tuned via a number of factors including: the material/mechanical properties of the polymer material used, and the geometric properties of the mesh structure including the type of mesh unit cell (e.g. Voronoi, Gyroid, etc.), the thickness of the mesh, the density of the mesh or infill density, and the beam thickness. These material and geometric properties are referred to herein as mesh parameters. In one example, a typical beam thickness may vary within the range of approximately 0.5 to 0.8 mm. The extrinsic properties of the 3D mesh structure MS can therefore be controlled through at least these mesh parameters to suit the specific application and user requirements.

In the illustrated embodiment, the collar body 110 includes a chin support region 110-r1, a neck support region 110-r2, and a fringe region 110-r3. The fringe region 110-r3 extends over a portion of the adjoining chest and shoulders of the user's torso to help stabilize and support the user's neck and chin in the predefined position. In particular, the fringe region 110-r3 is configured to distribute the mechanical load from the weight of the user's head exerted on the chin and neck support regions 110-r1, 110-r2 over an area of the torso, and can help to rectify left or right pronation of the user's neck, as based on clinical assessment of the user.

The outer shell portion 112 comprises one or more openings 1122 in the neck support region 110-r2 and/or fringe region 110-r3 for ventilation, comfort and/or flexibility, as shown. In the example shown, openings 1122-1 are provided in the fringe region 110-r3 for ventilation and flexibility, and an opening 1122-2 is provided in the neck region 110-r2 for the user's larynx. The 3D mesh structure MS can extend across the openings 1122 as shown, but this is not essential (e.g. see FIGS. 2A and 2B).

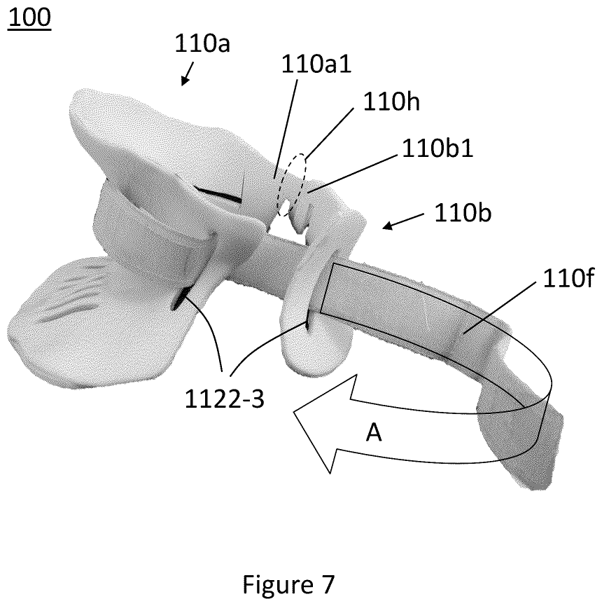

The neck collar 100 shown in FIGS. 1A and 1B comprises a split S at the rear of the collar body 110 for fitting to the user. In this embodiment, the collar 100 is a two-piece neck collar comprising a collar body 110 with a first (anterior) part 110a and a separate second (posterior) part 110b that is releasably connectable to the first part 110a via one or more fastening devices to permit transition of the collar body 110 between an open and closed position. The first part 110a of the collar body 110 is configured to extend around the anterior part of user's neck area with an inner surface 110s1 that conforms to the contours of the anterior part of the user's neck area. The second part 110b (shown in FIG. 1A is configured to extend across the split S of the first part 110a, and around the posterior part of the user's neck area, with a corresponding inner surface 110s2 that conforms to the contours of the posterior part of the user's neck area. FIGS. 1C and 1D show, respectively, side and rear views of the fitted neck collar 100 comprising the first and second parts 110a, 110b. In the illustrated embodiment, the first and second parts 110a, 110b are connectable at both sides of the collar 100 by one or more hook and loop (e.g. Velcro) fastening straps 110f which extend through openings 1122-3 provided in each side of the first and second parts 110a, 110b. In other embodiments, the first and second parts 110a, 110b are connected at one side by a hinge portion 110h to permit transition between an open and closed position and is releasably secured in the closed position by a fastening device 110f at the other side, such as a hook and loop fastening (see FIG. 7).

FIG. 2A shows a representative diagram of a custom neck collar 100 according to another embodiment of the invention. The neck collar 100 is similar to the neck collar 100 of FIG. 1A in that it comprises a customised collar body 110 with a solid outer shell portion 112 and an inner cushioning portion 114 formed of a 3D mesh structure MS, but in this example the 3D mesh structure MS comprises a gyroid lattice structure. The gyroid type mesh structure MS of the collar 100 is shown more clearly in FIG. 2B with the outer shell portion 112 omitted for clarity. Other lattice structures can be used, such as hexagonal lattice.

Different lattice structures may provide different mechanical and/or ventilation properties of the 3D mesh structure. The choice of which lattice structure to implement may thus depend on the specific design considerations for the patient, based on mechanical properties, specific weight of the final product, ventilation etc.

Although in FIGS. 1A-2B the 3D mesh structure MS is the same over the entire inner surface 110s of the collar body 110, this is not essential. In some embodiments, multiple different 3D mesh structures MS can be used in the same collar 100 to provide adequate mechanical and ventilation properties based on patient anatomical features and the associated properties of the particular mesh structure MS. For example, the inner cushioning portion 114 may comprise a plurality of regions, each region comprising a different 3D mesh structure. The inner cushioning portion 114 may comprise one or more first regions comprising a 3D mesh structure having a first lattice structure, and one or more second regions comprising a 3D mesh structure having a second lattice structure (not shown).

Further, although in FIGS. 1A-2B the 3D mesh structure MS forms or extends over the substantially the entire inner surface 110s of the collar body 110, this is not essential. For example, in the other embodiments, the 3D mesh structure 114 forms only part of the inner surface 110s of the collar body 110 (not shown). In such cases, the collar body 110 can comprise a single cushioning portion 114 formed by the 3D mesh structure MS that is ergonomically shaped to cover the main contact/pressure points of the neck and head area, or a plurality of discrete cushioning portions 114 provided at the main contact/pressure points of the neck and head area of the user. In general, the neck collar 100 of the invention is characterised by the 3D mesh structure MS forming at least a portion of the collar body 110. In yet other embodiments, the collar body 110 is formed entirely by the 3D mesh structure MS, described in more detail below.

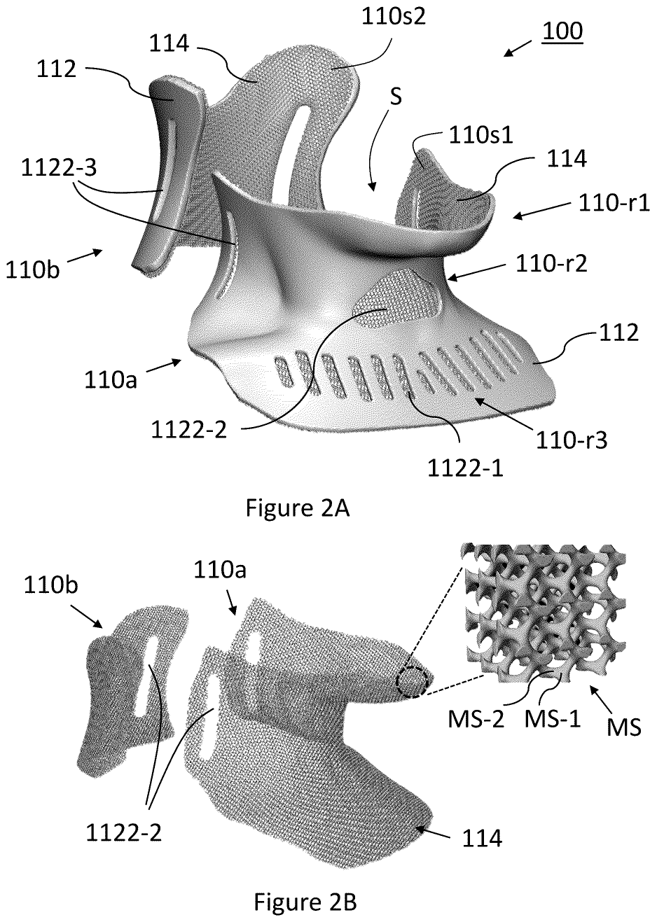

FIG. 3A shows a representative diagram of a first part 110a of a two-part custom neck collar 100 according to another embodiment of the invention, in which the collar body 110 is formed entirely by the 3D mesh structure MS. This provides an entirely breathable, lightweight, washable and hygienic custom neck collar 100. The all-mesh nature of the collar 100 also reduces the amount of material used to produce the collar 100 and associated manufacturing time and cost. Similar to the neck collar 100 of FIGS. 1A-2B, the collar body 110 comprises first and second parts 110a, 110b each having a unitary outer shell portion 112 and an inner cushioning portion 114, however in this case both are formed of a 3D mesh structure MS (note the second part 110b is omitted for clarity). FIG. 3B shows an enlarged view of the 3D mesh structure MS of the outer shell portion 112 and inner cushioning portion 114.

In preferred implementations, the properties (i.e. one or more mesh parameters) of the 3D mesh structure MS of the outer shell portion 112 and an inner cushioning portion 114 are different. Specifically, as described above, the 3D mesh structure MS of the inner cushioning portion 114 is configured to be substantially soft, compressible and/or pliable to provide cushioning, while the 3D mesh structure MS of the outer shell portion 112 is configured to be relatively hard and/or rigid to provide the required level of support and stability for the user's neck and head. The mechanical properties of the 3D mesh structures can be controlled via the mesh parameters as described above. In a preferred implementation, the outer shell portion 112 and the inner cushioning portion 114 are formed of different polymer materials. In one embodiment, the 3D mesh structure MS of the cushioning portion 114 is formed of or comprises a soft/elastomeric polymer material, such as a silicone rubber, while the outer shell portion 112 is formed of or comprises a relatively hard/rigid polymer material compared to the cushioning portion 114, such as nylon, acrylic styrene acrylonitrile, polylactic acid or polycarbonate. In another embodiment, both the 3D mesh structure MS of the inner cushioning portion 114 and the outer shell portion 112 are formed of or comprise a relatively hard/rigid polymer material, whereby the desired mechanical properties (i.e. substantially soft, compressible and/or pliable) of the cushioning portion 114 are produced by the geometric properties (mesh parameters) of the 3D mesh structure MS itself.

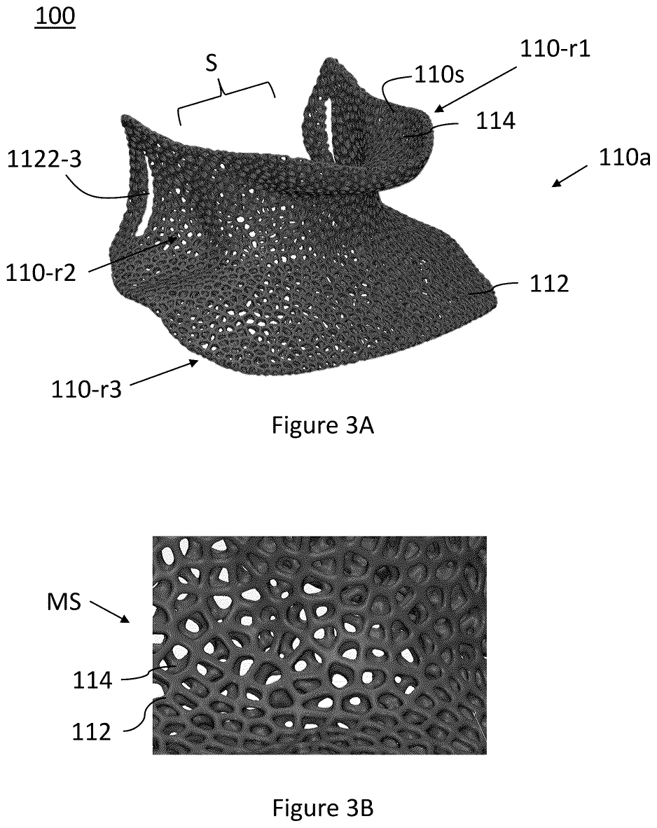

Other mesh parameters of the outer shell portion 112 and inner cushioning portion 114 can also or instead be varied to provide the desired properties (e.g. mesh density, infill density, beam width, unit cell type etc.). For example, in the illustrated embodiment of FIGS. 3A and 3B, both the outer shell portion 112 and inner cushioning portion 114 comprise the same type of lattice structure, which in this case is a Voronoi lattice structure. However, in other embodiments, the outer shell portion 112 and inner cushioning portion 114 can comprise different types of 3D mesh structures (not shown). As another example, FIG. 4 shows a representative diagram of the first part 110a of a custom all-mesh neck collar 100 formed with a lower density mesh structure MS.

In the illustrated examples of FIGS. 3A to 4, the 3D mesh structure is a Voronoi lattice structure, but this is not essential. The all-mesh collar 100 can also include one or more openings, such as the larynx opening 1122-2 (not shown). Further, in the illustrated embodiment the all-mesh collar 100 is configured as a two-part collar 100, as described above with reference to FIGS. 1A-1D. The first part 110a of the collar body 110 is illustrated in FIGS. 3A and 4 which comprises a split S at the back, and the collar 100 further comprises a second part 110b (not shown) which is connectable to the first part 110a at the split S for fitting to the user, in the same way as described above with reference to FIGS. 1A-1D.

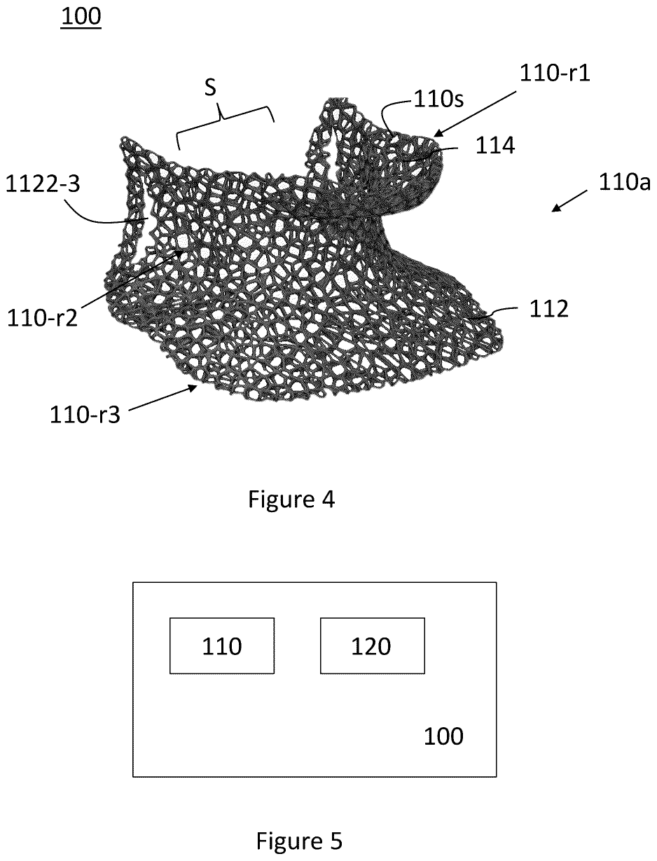

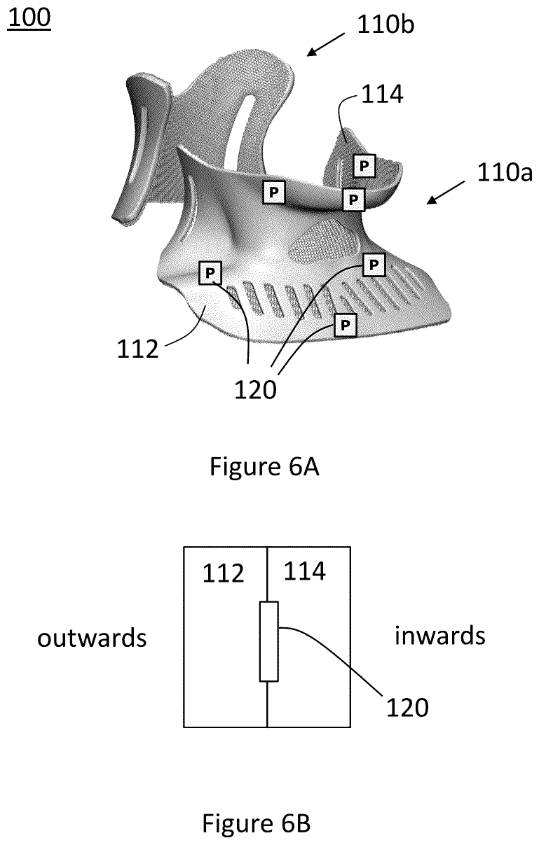

In various embodiments, the neck collar 100 also includes one or more sensors 120 integrated on or in the collar body 110 that are configured to monitor one or more parameters including at least one of: a force or pressure exerted on the collar by the user's neck, a temperature and/or a humidity in the collar 100. This is shown schematically in FIG. 5 and in more detail in FIGS. 6A and 6B.

FIG. 6A shows a representative diagram of a neck collar 100 illustrating the placement of sensors 120 at various landmarks on the collar 100. FIG. 6B shows a schematic sectional view of the collar 100 at a sensor location. As shown, the sensor 120 is positioned or embedded between the outer shell portion 112 and inner cushioning portion 114. Sensors 120 can be embedded during the 3D printing process.

FIG. 7 shows a representative diagram of a neck collar 100 according to an embodiment of the invention in which the first and second parts 110a, 110b of the collar body are connected at respective first sides/ends 110a1, 110b1 by a hinge point or portion 110h. In this example, the first and second parts 110a, 110b are physically connected (unitary) and the hinge point/portion 110h is formed by a constriction and/or a line of weakness at the point of connection of the first and second parts 110a, 110b. The first and second parts 110a, 110b are releasably connectable at the other side/end 110a2,110b2 by a hook and look fastening strap 110f which extends through openings 1122-3 provided in the respective second sides/ends 110a2, 110b2 as shown. In use, the collar 100 is fitted to the patient's neck in the open position and then transitioned to the closed position by pivoting the second part 110b about the hinge 110h so as to extend across the posterior portion of the neck. The collar is then releasably secured in the closed position by the fastening 110f, as indicated by arrow A in FIG. 7.

The custom neck collar 100 including the 3D mesh structure MS can be formed entirely by a 3D printing process. Alternatively, the custom neck collar 100 can be formed using a combination of 3D printing and subtractive manufacturing. For example, a multi-axis milling process (subtractive manufacturing) can be adapted to produce the open cell 3D mesh structure MS in the 3D printed inner cushioning portion 114. In this case, the 3D mesh structure MS of the cushioning portion 114 is preferably formed of or comprises a more rigid polymer material suitable for milling, such as polypropylene or urethane based materials.

The 3D printed neck collar 100 of the present invention is particularly suitable as a custom fit cervical orthosis for non-trauma patients who currently have no option but to use off-the-shelf collars with restrictive mobility. The neck collar 100 has an agronomic design to support comfort, hygiene and aesthetics, which are considered by user's/patients to be the most important features of a neck collar. Further, due to the integrated 3D mesh structure MS, the whole collar body 110 can be made relative thin compared to conventional neck collars, allowing it to be lightweight and seat within patient's garments. For example, the thickness of the collar body 110 (including the outer shell potion 112 and inner cushioning portion 114) can be as thin as approximately 5 mm, and may typically be varied in the range of between 5 to 8 mm or 5 to 10 mm. The open structure of mesh allows adequate ventilation, while the custom collar body can be biomechanically optimized to the patient's/user's condition, seats within patient's garments, improves comfort and thus compliance to manage their condition.

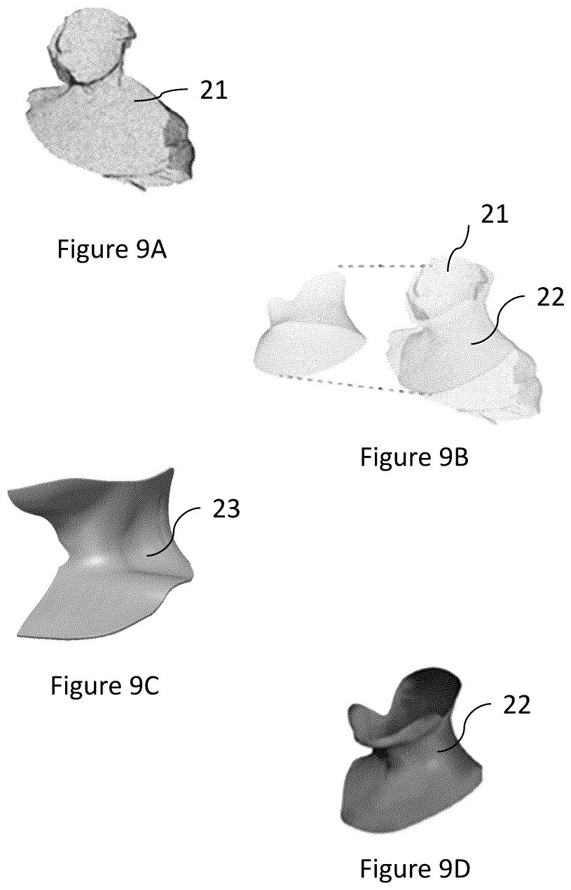

FIG. 8 shows a method 200 of manufacturing the neck collar 100 according to an embodiment of the invention. Step 210 comprises receiving or obtaining 3D surface scan data 21 of the user's neck area whilst their neck and head is in a predefined position. The scan should capture all the important anatomical landmarks, specifically around the chin region 110-r1. This provides the first working surface of the user's head and neck morphology for further processing in step 220. Example 3D scan data 21 obtained at step 210 is shown in FIG. 9A. In one implementation, this is achieved using a 3D scanner device, such as a handheld scanner. A suitable scanner is the ‘Artec EVA’. This particular 3D scanner has previously shown excellent agreement with high-resolution reference scans for torso and spinal imaging. The scan data 21 is optionally pre-processed and/or converted into a suitable format. For example, Artec Studio 12 Professional software can be used to process 3D surface scan data. The 3D surface scan data 21 can be validated by comparing real-world measurements to the 3D surface scan data, based on distances between anatomical landmarks. However, it will be appreciated that there is no restriction to the type of device used to obtain the 3D surface scan data 21. For example, in other implementations, an optical scanner or a smart phone or tablet device with a camera and a suitable 3D scanning application can be used to generate 3D scan data 21 of the user's neck and head area. The output of step 210 is preferably in .OBJ or .STL format.

Step 220 comprises generating an input solid model of the neck collar body 110 having an inner surface 110s that conforms to the contours of a user's neck area, based on the 3D surface scan data 21. In a preferred implementation, step 220 comprises generating a scan mesh that defines a 3D surface that conforms to the 3D surface scan data 21, relaxing the scan mesh away from the 3D surface scan data 21 to provide a comfortable inner surface 110 of the neck collar body 110, and extruding the surface defined by the relaxed scan mesh 22 to a desired thickness of the neck collar body 110 (to accommodate the thickness of the outer shell portion 112 and the inner cushioning portion 114) to produce the input solid model 23. Smoothing of the input solid collar model 23 and further identification of the pressure points can also be performed at this point. A representative image of a relaxed scan mesh 22 and resulting input solid model 23 is shown in FIGS. 9B and 9C respectively. In one implementation, step 220 is carried out using Houdini software (SideFX software, version 16.5). Optionally, the primitive surfaces of the 3D scan data 21 are first manually rectified and prepared for further processing. In this regard, manual cleaning, and smoothing of several skin- and scan related artifacts are to be performed. 3D Slicer (freeware) or equivalent software can be used to perform this task. The 3D scan data 21 is imported into the Houdini software, and a simple guide geometry, such as a cylinder, is positioned over the 3D scan data 21. This geometry is projected onto the 3D scan data 21. The projection process uses the point normals of the guide geometry to create ‘rays’ that extend in the direction of the normal. These normals are reversed to point inwards towards the 3D scan data geometry. If a given ‘ray’ collides with the 3D scan data 21, that point is moved to the collision point on the 3D scan data. This creates a scan mesh that conforms to the 3D scan data 21, with easily workable and modifiable geometry. As this projected geometry conforms absolutely to the 3D scan data 21, it is relaxed to move it away from the body to form a comfortable orthosis. Optionally, the relaxed scan mesh 22 is used for a calculation of the deformation energy to determine the required mechanical properties (e.g. stiffness parameters) of the collar body 110. FIG. 9D shows an example of the deformation energy mapped to the surface of the relaxed scan mesh 22.

Step 230 comprises generating an output solid model of a neck collar body 110 with at least a portion of the neck collar body 110 formed of the three-dimensional (3D) mesh structure MS, based on the input solid model 23 and one or more input mesh parameters. The one or more input mesh parameters include one or more of the following: a type of unit cell, material, an infill density, a coverage and/or perimeter of the mesh structure, and a width of the beams MS-2 of the mesh structure. In step 230, a solid surface of the input model 23 having a predefined thickness is used as an input (in .OBJ format) to a computer program configured to produce the mesh structure. The thickness of the solid surface defines the thickness of the 3D mesh structure MS of the cushioning portion 114. This effectively divides the input model into two parts, the outer shell portion 112 and the inner cushioning portion 114. By way of example, the computer program can be implemented using any software platform which allows scripting, such as Rhinoceros, or Materialise 3Matic, few notable amongst many. In an example implementation, the following mesh parameters are used as input to initiation of the mesh creation from the input solid model: (a) type of unit cell e.g., Voronoi lattice, gyroid, hexagonal lattice etc., (b) packing density distribution i.e., how much infill density is required for the 3D mesh structures, and (c) thickness of each beam MS-2 of a 3D unit cell of the mesh structure. Based on these input mesh parameters, the solid surface is converted into the 3D mesh structure MS of the collar body 110, and the output solid model of the neck collar body 110 including the outer shell portion 112 and the integrated inner cushioning portion 114 is generated as an output (in . STL format, preferably). In embodiments where only the inner cushioning portion 114 is formed as a 3D mesh structure MS, the solid surface represents a layer or portion of the input solid model of the collar 110, and the remainder of the input model is used as the solid outer shell portion 112. After development of the mesh structure MS, the two parts of the model (the outer shell portion 112 and the mesh structure MS of the inner cushioning portion 114) can be joined together to provide the output solid model and manufactured as one integrated part using the required materials. In embodiments where outer shell portion 112 and the inner cushioning portion 114 are both formed as a 3D mesh structure MS, the solid surface solid surface is converted into the 3D mesh structure MS of the inner cushioning portion 114, and remainder of the input model is converted into the 3D mesh structure MS of the outer shell portion 114 (using their respective mesh parameters).

Step 240 comprises fabricating, using a 3D printer, the custom neck collar body 110 based on the solid model produced in step 230 using the desired 3D printable materials. In step 240, the output of step 230 is given as an input to the 3D printing software of the 3D printer. The printability of the output solid model may be checked prior to proceeding for execution of the 3D printing to identify whether the output of the step 230 is ready to be printed or warrants further modification.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

What is claimed is:

1-26. (canceled)

27. A custom neck collar for supporting the neck and head of a user in a pre-defined position, comprising:
a three-dimensional (3D) printed collar body having an inner surface that conforms to the contours of a user's neck area, and wherein at least a portion of the collar body is formed of or comprises a 3D mesh structure.

28. The collar of claim 27, wherein the at least a portion comprising the 3D mesh structure forms at least part of the inner surface of the collar body for cushioning.

29. The collar of claim 27, wherein the at least a portion comprising the 3D mesh structure forms the entire inner surface of the collar body for cushioning.

30. The collar of claim 27, wherein the collar body comprises a outer shell portion and at least one inner cushioning portion comprised of the 3D mesh structure, wherein the at least one inner cushioning portion is unitary with the outer shell portion.

31. The collar of claim 30, wherein the 3D mesh structure of the at least one inner cushioning portion is formed of or comprises a polymer material and is configured to be relatively soft, compressible and/or pliable compared to the outer shell portion.

32. The collar of claim 30, wherein the outer shell portion is formed of or comprises a polymer material and is configured to be substantially rigid to restrict movement of the neck and head from the predefined position.

33. The collar of claim 30, wherein the outer shell portion is formed of or comprises a polymer material and is configured to be substantially flexible to permit limited movement of the neck and head from the predefined position.

34. The collar of claim 30, wherein the outer shell portion comprises one or more openings for ventilation.

35. The collar of claim 27, wherein the entire collar body is formed of or comprises the 3D mesh structure.

36. The collar of claim 30, wherein the outer shell portion is also comprised of the 3D mesh structure; wherein the 3D mesh structure of the outer shell portion and the 3D mesh structure of the at least one inner cushioning portion are defined by a plurality of mesh parameters, and wherein at least one mesh parameter of the outer shell portion is different to the corresponding at least one mesh parameter of the at least one inner cushioning portion.

37. The collar of claim 30, wherein the outer shell portion is a solid outer shell portion without a 3D mesh structure.

38. The collar of claim 27, wherein the 3D mesh structure is formed of or comprises an antibacterial material.

39. The collar of claim 27, wherein the collar body is configured to be:
substantially flexible to permit limited movement of the neck and head from the predefined position; or
substantially rigid to restrict movement of the neck and head from the predefined position.

40. The collar of claim 27, wherein the 3D mesh structure is an open cell structure.

41. The collar of claim 40, wherein the 3D mesh structure comprises one of the following geometries: a gyroid lattice structure, a hexagonal lattice structure, a Voronoi lattice structure, a rhombohedral lattice structure, a dodecahedron lattice structure, and a Schwartz lattice structure.

42. The collar of claim 27, further comprising one or more sensors integrated on or in the collar body, wherein the one or more sensors are configured to monitor one or more of: a force or pressure exerted on the collar by the user's neck, a temperature and/or a humidity in the collar.

43. The collar of claim 27, wherein the collar body comprises an anterior first portion and a second portion that are connectable to permit transition of the collar between an open and closed position for fitting to a user, wherein the first and second parts are:
(i) connected at a first side of the collar body by a hinge or hinge portion and connectable at a second side of the collar body by one or more releasable fastening devices; or
(ii) connectable at first and second sides of the collar body by one or more releasable fastening devices.

44. The collar of claim 43 parts (i) or (ii), wherein the one or more releasable fastening devices is or comprises one or more of the following: a hook and loop fastener, a magnetic fastener, and a mechanical fastener.

45. The collar of claim 27, wherein the collar does not comprise additional cushioning material or a separate padded liner.

46. The collar of claim 27, wherein the collar body is formed of or comprises one or more polymer materials.
